(19) United States
(12) Patent Application Publication
(10) Pub. No.: US 2020/0029856 A1
(43) Pub. Date: Jan. 30, 2020

(54) Needle Assembly Having an Optical Sensor for Improved Placement Within a Patient

(71) Applicant: Avent, Inc., Alpharetta, GA (US)

(72) Inventors: Shiva Sharareh, Laguna Niguel, CA (US); Thomas D. Mina, Newport Beach, CA (US)

(21) Appl. No.: 16/045,911

(22) Filed: Jul. 26, 2018

(51) Int. Cl. A61B 5/06; A61B 5/00; A61B 8/08; B33Y 10/00; B33Y 80/00

(52) U.S. Cl. CPC A61B 5/066; A61B 5/0084; A61B 5/0075; A61B 2562/12; B33Y 10/00; B33Y 80/00; A61B 8/0841

(57) ABSTRACT

A needle assembly for an ultrasound imaging system includes a needle having a proximal end and a distal end. The distal end is adapted to be inserted into a patient. The needle assembly also includes an optical sensor assembly secured to the distal end of the needle. The optical sensor assembly has a field of vision that includes the distal end of the needle and an environment surrounding the distal end of the needle as the needle is inserted into the patient towards a target site. In addition, the needle assembly includes a controller communicatively coupled to the optical sensor assembly. Thus, the controller is configured to receive and process one or more sensor signals from the optical sensor assembly in real-time.

### FIELD OF THE INVENTION

The present invention relates to generally to needle assemblies for use in nerve block procedures, and more particularly, to a needle assembly having an optical sensor configured to provide improved needle placement within a patient.

### BACKGROUND

Detection of anatomical objects using medical imaging is an essential step for many medical procedures, such as regional anesthesia nerve blocks, and is becoming the standard in clinical practice to support diagnosis, patient stratification, therapy planning, intervention, and/or follow-up. Various systems based on traditional approaches exist for anatomical detection and tracking in medical images, such as computed tomography (CT), magnetic resonance (MR), ultrasound, and fluoroscopic images.

For example, ultrasound imaging systems utilize sound waves with frequencies higher than the upper audible limit of human hearing. Further, ultrasound imaging systems are widely used in medicine to perform both diagnosis and therapeutic procedures. In such procedures, sonographers perform scans of a patient using a hand-held probe or transducer that is placed directly on and moved over the patient.

Accurate needle placement is incredibly important to the success of a nerve block procedure, and current ultrasound methods can often prove challenging in providing the optimal needle placement. As such, accurate needle placement often affects the overall efficacy of a nerve block procedure, thereby increasing time of the procedure and decreasing patient satisfaction. However, accurate needle placement is often extremely difficult to achieve due to a multitude of factors. For example, ultrasound technologies are oftentimes not the most effective tools for nerve visualization and needle guidance, as such systems rely on a granular images to provide physicians with the anatomical features they need to visualize in order to make the proper placement. In addition, in many instances, the physician must use one hand to guide the needle while using the other hand to hold the ultrasound probe.

Accordingly, the present disclosure is directed to a needle assembly having an optical sensor that addresses the aforementioned issues.

### SUMMARY OF THE INVENTION

Objects and advantages of the invention will be set forth in part in the following description, or may be obvious from the description, or may be learned through practice of the invention.

In one aspect, the present invention is directed to a needle assembly for an ultrasound imaging system. The needle assembly includes a needle having a proximal end and a distal end. The distal end is adapted to be inserted into a patient. The needle assembly also includes an optical sensor assembly secured to the distal end of the needle. The optical sensor assembly has a field of vision that includes the distal end of the needle and an environment surrounding the distal end of the needle as the needle is inserted into the patient towards a target site. In addition, the needle assembly includes a controller communicatively coupled to the optical sensor assembly. Thus, the controller is configured to receive and process one or more sensor signals from the optical sensor assembly in real-time.

In one embodiment, the optical sensor assembly may include one or more optical sensors printed to the distal end of the needle via an additive manufacturing process. For example, in particular embodiments, the additive manufacturing process may include fused deposition modeling, stereolithography, digital light processing, metal wire transfer, electron beam melting, inertial welding, powder nozzle laser deposition, directed energy deposition, laser cladding, cold spray deposition, directed energy deposition, powder bed fusion, material extrusion, direct metal laser sintering, direct metal laser melting, cold metal transfer, or any other suitable additive manufacturing process.

In another embodiment, the controller is further configured to generate one or more images comprising a real-time view of the environment surrounding the distal end of the needle using the one or more sensor signals. In particular embodiments, for example, the generated image(s) may include one or more spectral images. In such embodiments, the needle assembly may also include a display for displaying the spectral image(s) to a user.

In further embodiments, the controller may also be configured to provide haptic feedback to a user as the distal end of the needle approaches the target site of the patient.

In additional embodiments, each of the optical sensor(s) may include a receiver for receiving the one or more sensor signals and a transmitter for sending the one or more spectral images to the display.

In several embodiments, the optical sensor assembly may include a plurality of optical sensors positioned adjacent to each other at the distal end of the needle. In yet another embodiment, each of the optical sensor(s) may have a predetermined thickness ranging from about 0.01 millimeters (mm) to about 0.05 mm.

In another aspect, the present disclosure is directed to a method for manufacturing a needle assembly of an ultrasound imaging system. The method includes providing a needle having a proximal end and a distal end. The distal end of the needle is adapted to be inserted into a patient. The method also includes printing an optical sensor assembly onto the distal end of the needle via an additive manufacturing process. As such, the optical sensor assembly has a field of vision that includes the distal end of the needle and an environment surrounding the distal end of the needle as the needle is inserted into the patient towards a target site. Further, the method includes communicatively coupling a controller to the optical sensor assembly. Thus, the controller is configured to receive and process one or more sensor signals from the optical sensor assembly in real-time.

In one embodiment, the step of printing the optical sensor assembly at the distal end of the needle via the additive manufacturing process may include printing one or more optical sensors onto an outer circumference of the distal end of the needle. In such embodiments, the step of printing one or more optical sensors onto an outer circumference of the distal end of the needle may include printing one or more layers of material onto the outer circumference of the distal end of the needle to form the one or more optical sensors.

In another embodiment, the step of printing one or more optical sensors onto the outer circumference of the distal end of the needle may include printing a plurality of optical sensors onto the outer circumference of the distal end of the needle. In such embodiments, each of the plurality of optical sensors may include a receiver for receiving the one or more sensor signals and a transmitter for sending the one or more spectral images to the display. In further embodiments, the method may include printing the plurality of optical sensors adjacent to each other at the distal end of the needle. It should also be understood that the method may further include any of the additional steps and/or features as described herein.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of one embodiment of an imaging system according to the present disclosure;

FIG. 2 illustrates a block diagram one of embodiment of a controller of an imaging system according to the present disclosure;

FIG. 3 illustrates a schematic diagram of one embodiment of a needle assembly according to the present disclosure;

FIG. 4 illustrates a detailed view of the distal end of the needle assembly of FIG. 3, particularly illustrating the optical sensor assembly printed at the distal end of the needle assembly; and

FIG. 5 illustrates a flow diagram of one embodiment of a method for manufacturing a needle assembly of an ultrasound imaging system according to the present disclosure.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

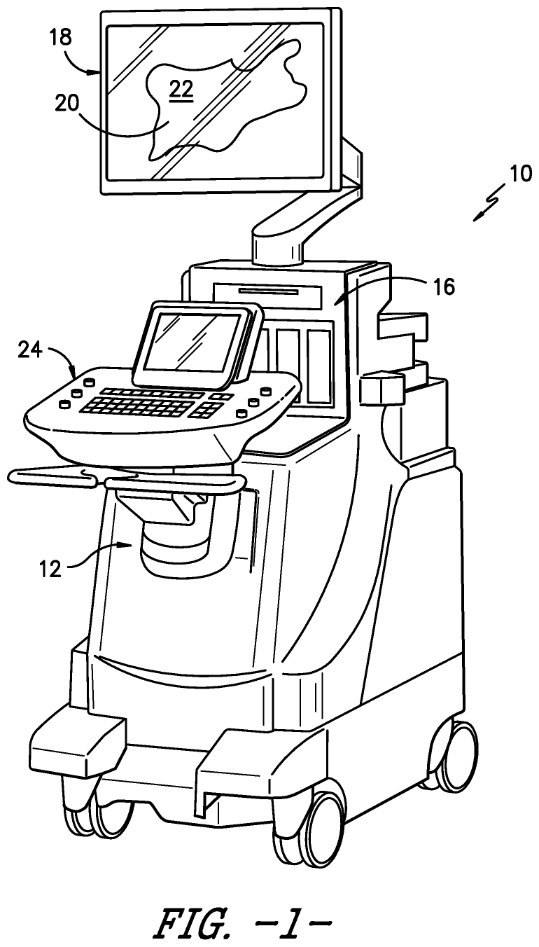

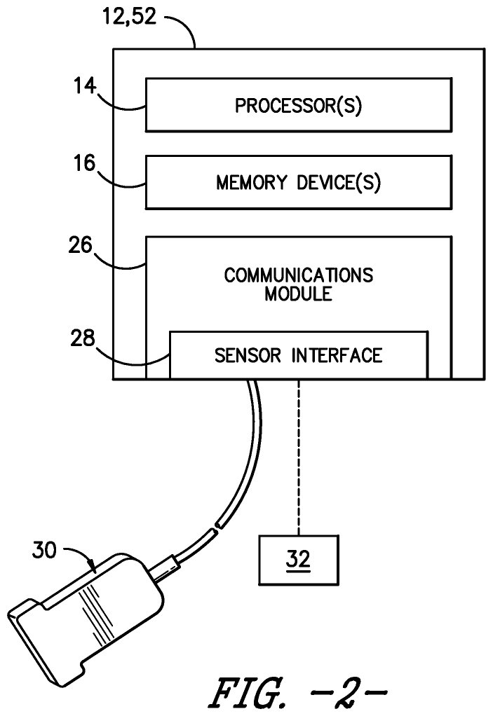

Referring now to the drawings, FIGS. 1 and 2 illustrate a medical imaging system 10 for scanning, identifying, and navigating anatomical objects of a patient according to the present disclosure. As used herein, the anatomical object(s) 22 and surrounding tissue described herein may include any anatomical structure and/or surrounding tissue of a patient. For example, in one embodiment, the anatomical object(s) 22 may include one or more nerves or nerve bundles. More specifically, in another embodiment, the anatomical object(s) 22 may include an interscalene brachial plexus of the patient, which generally corresponds to the network of nerves running from the spine, formed by the anterior rami of the lower four cervical nerves and first thoracic nerve. As such, the surrounding tissue of the brachial plexus generally corresponds to the sternocleidomastoid muscle, the middle scalene muscle, the anterior scalene muscle, and/or similar.

It should be understood, however, that the system and method of the present disclosure may be further used for any variety of medical procedures involving any anatomical structure in addition to those relating to the brachial plexus. For example, the anatomical object(s) 22 may include upper and lower extremities, as well as compartment blocks. More specifically, in such embodiments, the anatomical object(s) 22 of the upper extremities may include interscalene muscle, supraclavicular muscle, infraclavicular muscle, and/or axillary muscle nerve blocks, which all block the brachial plexus (a bundle of nerves to the upper extremity), but at different locations. Further, the anatomical object(s) 22 of the lower extremities may include the lumbar plexus, the fascia Iliac, the femoral nerve, the sciatic nerve, the abductor canal, the popliteal, the saphenous (ankle), and/or similar. In addition, the anatomical object(s) 22 of the compartment blocks may include the intercostal space, transversus abdominus plane, and thoracic paravertebral space, and/or similar.

In addition, as shown, the imaging system 10 may correspond to an ultrasound imaging system or any other suitable imaging system that can benefit from the present technology. Thus, as shown, the imaging system 10 may generally include a controller 12 having one or more processor(s) 14 and associated memory device(s) 16 configured to perform a variety of computer-implemented functions (e.g., performing the methods and the like and storing relevant data as disclosed herein), as well as a user display 18 configured to display an image 20 of an anatomical object 22 or the surrounding tissue to an operator. In addition, the imaging system 10 may include a user interface 24, such as a computer and/or keyboard, configured to assist a user in generating and/or manipulating the user display 18.

Additionally, as shown in FIG. 2, the processor(s) 14 may also include a communications module 26 to facilitate communications between the processor(s) 14 and the various components of the imaging system 10, e.g. any of the components of FIG. 1. Further, the communications module 26 may include a sensor interface 28 (e.g., one or more analog-to-digital converters) to permit signals transmitted from one or more probes (e.g. an ultrasound transducer 30 or an optical sensor assembly 32 described herein) to be converted into signals that can be understood and processed by the processor(s) 14. It should be appreciated that the ultrasound transducer 30 and/or the optical sensor assembly 32 may be communicatively coupled to the communications module 26 using any suitable means. For example, as shown in FIG. 2, the sensors 30, 32 may be coupled to the sensor interface 28 via a wired connection. However, in other embodiments, the sensors 30, 32 may be coupled to the sensor interface 28 via a wireless connection, such as by using any suitable wireless communications protocol known in the art. As such, the processor(s) 14 may be configured to receive one or more sensor signals from the sensors 30, 32.

As used herein, the term “processor” refers not only to integrated circuits referred to in the art as being included in a computer, but also refers to a controller, a microcontroller, a microcomputer, a programmable logic controller (PLC), an application specific integrated circuit, a field-programmable gate array (FPGA), and other programmable circuits. The processor(s) 14 is also configured to compute advanced control algorithms and communicate to a variety of Ethernet or serial-based protocols (Modbus, OPC, CAN, etc.). Furthermore, in certain embodiments, the processor(s) 14 may communicate with a server through the Internet for cloud computing in order to reduce the computation time and burden on the local device. Additionally, the memory device(s) 16 may generally comprise memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) 16 may generally be configured to store suitable computer-readable instructions that, when implemented by the processor(s) 14, configure the processor(s) 14 to perform the various functions as described herein.

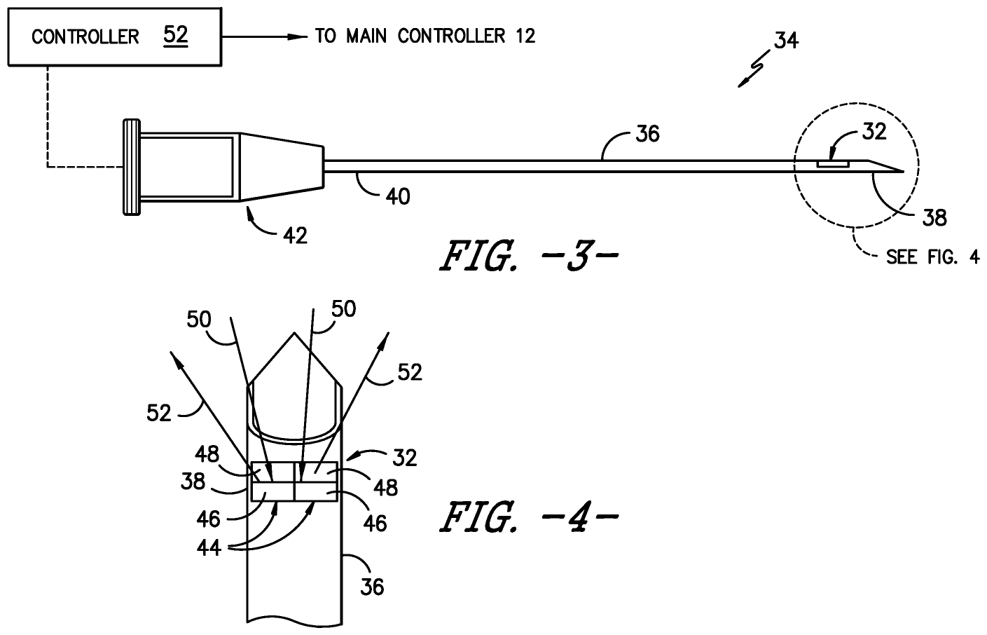

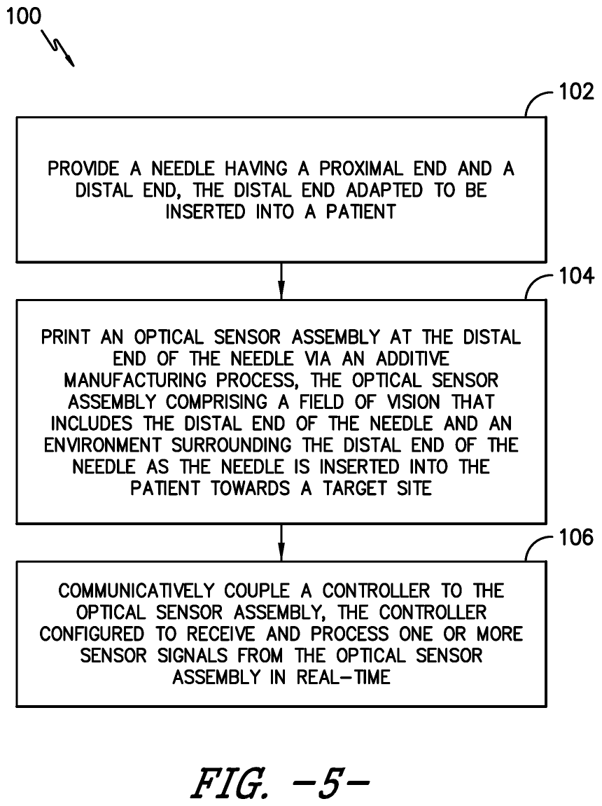

Referring to FIGS. 3 and 4, various views of one embodiment of a needle assembly 34 for the ultrasound imaging system 10 according the present disclosure are illustrated. For example, FIG. 3 illustrates a schematic diagram of one embodiment of the needle assembly 34 for the ultrasound imaging system 10 according to the present disclosure. More specifically, as shown, the needle assembly 34 includes a needle 36 having a proximal end 40 and a distal end 38 adapted to be inserted into a patient, an optical sensor assembly 32, and a controller 52 communicatively coupled to the optical sensor assembly 32. As such, the needle assembly 34 is configured to enhance visualization of the needle tip during a medical procedure, such as a nerve block procedure, via the optical sensor assembly 32 positioned at the distal end 38 of the needle 36. Moreover, as shown, the needle 36 may also include a needle hub 42 at its proximal end 40. In such embodiments, the optical sensor assembly 32 may be communicatively coupled to the controller 12 via the needle hub 42.

Referring particularly to FIG. 4, the optical sensor assembly 32 may further include one or more optical sensors 44 printed to the distal end 38 of the needle 36 via an additive manufacturing process. For example, as shown, each of the optical sensor(s) 44 may include a receiver 46 for receiving one or more sensor signals 50 relating to the tissue environment and a transmitter 48 for sending the processed signals (e.g. spectral images) to the controller 12 (or the display 18). In addition, as shown in the illustrated embodiment, the optical sensor assembly 32 may include a plurality of optical sensors 44 positioned adjacent to each other at the distal end 38 of the needle 36.

The additive manufacturing process described herein may include any of the following: fused deposition modeling, stereolithography, digital light processing, metal wire transfer, electron beam melting, inertial welding, powder nozzle laser deposition, directed energy deposition, laser cladding, cold spray deposition, directed energy deposition, powder bed fusion, material extrusion, direct metal laser sintering, direct metal laser melting, cold metal transfer, or any other suitable additive manufacturing process. By using additive manufacturing, the optical sensors 44 can be printed at the distal end 38 of the needle 36 in thin layers so as not to disturb the overall efficacy of the needle 36 in puncturing the necessary tissue of the patient. For example, in one embodiment, each of the optical sensor(s) 44 may have a predetermined thickness ranging from about 0.01 millimeters (mm) to about 0.05 mm. As used herein, terms of degree, such as “about,” are meant to encompass a range of +/−10% from the value set forth.

Accordingly, the optical sensor assembly 32 of the present disclosure has a field of vision that includes the distal end 38 of the needle 36 and the environment surrounding the distal end 38 of the needle 36, e.g. as the needle 36 is inserted into the patient towards a target site. Thus, the controller 52 is configured to receive and process the sensor signal(s) 50 in real-time. In addition, the controller 52 is configured to generate one or more images that display a real-time view of the environment surrounding the distal end 38 of the needle 36 using the sensor signals 50. In particular embodiments, for example, the generated image(s) may include one or more spectral images. As such, the controller 52 is configured to distinguish between spectral changes in the environment to allow for easier needle guidance prior to final placement of the needle 36. Thus, in one embodiment, the ability to visualize both the nerve and the needle tip allows for optimal guidance and placement of the needle 36, thereby resulting in improved drug delivery throughout the procedure with minimal reliance on the ultrasound images when placing the needle 16. In such embodiments, the controller 52 may communicate with the main controller 12 such that the display 18 of the ultrasound imaging system 10 can display the spectral image(s) to a user. It should also be understood that the controller 52 may be similarly configured to controller 12.

Additionally, the controller 52 may be configured to generate haptic feedback (e.g. through the needle 36 and/or the needle hub 42) via vibration, pulses, etc. to indicate to a user when the needle 36 is a certain distance away from the target nerve.

Referring now to FIG. 5, a flow diagram of one embodiment of a method 100 for manufacturing a needle assembly of an ultrasound imaging system is illustrated. In general, the method 100 will be described herein with reference to the ultrasound imaging system 10 shown in FIGS. 1 and 2. However, it should be appreciated that the disclosed method 100 may be implemented with imaging systems having any other suitable configurations and/or within systems having any other suitable system configuration. In addition, although FIG. 5 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown at 102, the method 100 includes providing the needle 36 with its distal and proximal ends 38, 40. As shown at 104, the method 100 includes printing the optical sensor assembly 32 onto the distal end 38 of the needle 36 via an additive manufacturing process (such as any of the additive processes described herein). Thus, once printed, the optical sensor assembly 32 has a field of vision that includes the distal end 38 of the needle 36 and the environment surrounding the distal end 38 as the needle 36 is inserted into the patient towards a target site. For example, in one embodiment, the optical sensor assembly 32 (which may include one optical sensor 44 or a plurality of optical sensors 44) may be printed at the distal end 38 of the needle 36 by printing one or more optical sensors 44 onto an outer circumference of the distal end 38 of the needle 36. In such embodiments, the additive manufacturing process may include printing one or more thin layers of material onto the outer circumference of the distal end 38 of the needle 36 to form the one or more optical sensors 44.

Still referring to FIG. 5, as shown at 106, the method 100 may also include communicatively coupling the controller 12 to the optical sensor assembly 32. Thus, as mentioned, the controller 52 is configured to receive and process one or more sensor signals 50 from the optical sensor assembly 32 in real-time.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A needle assembly for an ultrasound imaging system, the needle assembly comprising:
a needle comprising a proximal end and a distal end, the distal end adapted to be inserted into a patient;
an optical sensor assembly secured to the distal end of the needle, the optical sensor assembly comprising a field of vision that includes the distal end of the needle and an environment surrounding the distal end of the needle as the needle is inserted into the patient towards a target site; and
a controller communicatively coupled to the optical sensor assembly, the controller configured to receive and process one or more sensor signals from the optical sensor assembly in real-time.

2. The needle assembly of claim 1, wherein the optical sensor assembly comprises one or more optical sensors printed to the distal end of the needle via an additive manufacturing process.

3. The needle assembly of claim 2, wherein the additive manufacturing process comprises at least one of fused deposition modeling, stereolithography, digital light processing, metal wire transfer, electron beam melting, inertial welding, powder nozzle laser deposition, directed energy deposition, laser cladding, cold spray deposition, directed energy deposition, powder bed fusion, material extrusion, direct metal laser sintering, direct metal laser melting, or cold metal transfer.

4. The needle assembly of claim 2, wherein the controller is further configured to generate one or more images comprising a real-time view of the environment surrounding the distal end of the needle using the one or more sensor signals.

5. The needle assembly of claim 4, wherein the one or more images comprise one or more spectral images.

6. The needle assembly of claim 5, further comprising a display for displaying the one or more spectral images to a user.

7. The needle assembly of claim 6, wherein each of the one or more optical sensors comprises a receiver for receiving the one or more sensor signals and a transmitter for sending the one or more spectral images to the display.

8. The needle assembly of claim 2, wherein the optical sensor assembly further comprises a plurality of optical sensors positioned adjacent to each other at the distal end of the needle.

9. The needle assembly of claim 2, wherein each of the one or more optical sensors comprises a predetermined thickness ranging from about 0.01 millimeters (mm) to about 0.05 mm.

10. The needle assembly of claim 1, wherein the controller is configured to provide haptic feedback to a user as the distal end of the needle approaches the target site of the patient.

11. A method for manufacturing a needle assembly of an ultrasound imaging system, the method comprising:
providing a needle having a proximal end and a distal end, the distal end adapted to be inserted into a patient;
printing an optical sensor assembly at the distal end of the needle via an additive manufacturing process, the optical sensor assembly comprising a field of vision that includes the distal end of the needle and an environment surrounding the distal end of the needle as the needle is inserted into the patient towards a target site; and
communicatively coupling a controller to the optical sensor assembly, the controller configured to receive and process one or more sensor signals from the optical sensor assembly in real-time.

12. The method of claim 11, wherein printing the optical sensor assembly at the distal end of the needle via the additive manufacturing process further comprises printing one or more optical sensors onto an outer circumference of the distal end of the needle.

13. The method of claim 12, wherein printing one or more optical sensors onto the outer circumference of the distal end of the needle further comprises printing one or more layers of material onto the outer circumference of the distal end of the needle to form the one or more optical sensors.

14. The method of claim 12, wherein printing one or more optical sensors onto the outer circumference of the distal end of the needle further comprises printing a plurality of optical sensors onto the outer circumference of the distal end of the needle.

15. The method of claim 14, wherein each of the plurality of optical sensors comprises a receiver for receiving the one or more sensor signals and a transmitter for sending the one or more spectral images to the display.

16. The method of claim 14, further comprising printing the plurality of optical sensors adjacent to each other at the distal end of the needle.

17. The method of claim 14, wherein the plurality of optical sensors each comprise a predetermined thickness ranging from about 0.01 millimeters (mm) to about 0.05 mm.

18. The method of claim 11, wherein the additive manufacturing process comprises at least one of fused deposition modeling, stereolithography, digital light processing, metal wire transfer, electron beam melting, inertial welding, powder nozzle laser deposition, directed energy deposition, laser cladding, cold spray deposition, directed energy deposition, powder bed fusion, material extrusion, direct metal laser sintering, direct metal laser melting, or cold metal transfer.

19. The method of claim 11, wherein the controller is further configured to generate one or more spectral images comprising a real-time view of the environment surrounding the distal end of the needle using the one or more sensor signals.

20. The method of claim 11, wherein the controller is configured to provide haptic feedback to a user as the distal end of the needle approaches the target site of the patient.
